SEQUENCE LISTING (20 sequences)

SEQ ID NO: 1 - protein, 15 aa, Artificial Sequence
  Synthetic peptide
GGGGSGGGGSGGGGS

SEQ ID NO: 2 - protein, 8 aa, Artificial Sequence
  Synthetic peptide
GGGGGGGG

SEQ ID NO: 3 - protein, 6 aa, Artificial Sequence
  Synthetic peptide
GGGGGG

SEQ ID NO: 4 - protein, 15 aa, Artificial Sequence
  Synthetic peptide
EAAAKEAAAKEAAAK

SEQ ID NO: 5 - protein, 15 aa, Artificial Sequence
  Synthetic peptide
  misc_feature (6)..(15): Either or both of the “Glu Ala Ala Ala Lys” may be present or absent
EAAAKEAAAKEAAAK

SEQ ID NO: 6 - protein, 46 aa, Artificial Sequence
  Synthetic peptide
AEAAAKEAAAKEAAAKEAAAKALEAEAAAKEAAAKEAAAKEAAAKA

SEQ ID NO: 7 - protein, 5 aa, Artificial Sequence
  Synthetic peptide
GGGGS

SEQ ID NO: 8 - protein, 5 aa, Artificial Sequence
  Synthetic peptide
PAPAP

SEQ ID NO: 9 - protein, 12 aa, Artificial Sequence
  Synthetic peptide
AEAAAKEAAAKA

SEQ ID NO: 10 - protein, 20 aa, Artificial Sequence
  Synthetic peptide
  misc_feature (6)..(20): Two or Three  “Gly Gly Gly Gly Ser” may be absent
GGGGSGGGGSGGGGSGGGGS

SEQ ID NO: 11 - protein, 34 aa, Artificial Sequence
  Synthetic peptide
  misc_feature (11)..(34): Any one or all “Ala Pro” may be present or absent
APAPAPAPAPAPAPAPAPAPAPAPAPAPAPAPAP

SEQ ID NO: 12 - protein, 17 aa, Artificial Sequence
  Synthetic peptide
  misc_feature (9)..(10): Factor XIa/FVIIa sensitive cleavage
VSQTSKLTRAETVFPDV

SEQ ID NO: 13 - protein, 6 aa, Artificial Sequence
  Synthetic peptide
  misc_feature (1)..(6): Matrix metalloprotease-1 sensitive cleavage sequence
  misc_feature (3)..(4): Protease sensitive cleavage site
PLGLWA

SEQ ID NO: 14 - protein, 6 aa, Artificial Sequence
  Synthetic peptide
  misc_feature (3)..(4): Protease sensitive cleavage site
RVLAEA

SEQ ID NO: 15 - protein, 10 aa, Artificial Sequence
  Synthetic peptide
  misc_feature (6)..(7): Protease sensitive cleavage site
EDVVCCSMSY

SEQ ID NO: 16 - protein, 8 aa, Artificial Sequence
  Synthetic peptide
  misc_feature (1)..(8): Matrix metalloprotease-1 sensitive cleavage sequence
  misc_feature (6)..(7): Protease sensitive cleavage site
GGIEGRGS

SEQ ID NO: 17 - protein, 10 aa, Artificial Sequence
  Synthetic peptide
  misc_feature (1)..(10): HIV PR (HIV-1 protease)
  misc_feature (7)..(8): Protease sensitive cleavage site
TRHRQPRGWE

SEQ ID NO: 18 - protein, 10 aa, Artificial Sequence
  Synthetic peptide
  misc_feature (1)..(10): NS3 protease (HCV protease)
  misc_feature (7)..(8): Protease sensitive cleavage site
AGNRVRRSVG

SEQ ID NO: 19 - protein, 9 aa, Artificial Sequence
  Synthetic peptide
  misc_feature (1)..(9): Factor Xa sensitive cleavage
  misc_feature (7)..(8): Protease sensitive cleavage site
  misc_feature (8)..(9): Protease sensitive cleavage site
RRRRRRRRR

SEQ ID NO: 20 - protein, 4 aa, Artificial Sequence
  Synthetic peptide
  misc_feature (1)..(4): Furin sensitive cleavage
  misc_feature (4)..(4): Protease sensitive cleavage site
GFLG

(19) United States
(12) Patent Application Publication
(10) Pub. No.: US 2018/0140724 A1
(43) Pub. Date: May 24, 2018

(54) Tumor Deliverable Iron and Protein Synthesis Inhibitors as a New Class of Drugs for the Diagnosis and Treatment of Cancer

(71) Applicants: Changchun Deng, Jericho, NY (US); Mark Lipstein, New York, NY (US); Owen A. O'Connor, Scarsdale, NY (US)

(72) Inventors: Changchun Deng, Jericho, NY (US); Mark Lipstein, New York, NY (US); Owen A. O'Connor, Scarsdale, NY (US)

(73) Assignee: The Trustees of Columbia University in the City of New York, New York, NY (US)

(21) Appl. No.: 15/576,306

(22) PCT Filed: May 27, 2016
(86) PCT No.: PCT/US16/34701

§ 371 Date: Nov. 22, 2017

Related U.S. Application Data
(60) Provisional application No. 62/166,837, filed on May 27, 2015.

(51) Int. Cl. A61K 49/16; A61K 47/68; C07K 16/32; C07K 16/30; C07K 16/28; A61P 35/00

(52) U.S. Cl. CPC A61K 49/16; A61K 47/6849; A61K 47/6851; A61K 47/6889; A61P 35/00; C07K 16/32; C07K 16/30; C07K 16/2887; A61K 47/6855

(57) ABSTRACT

Tumor deliverable iron (TDI) drugs and pharmaceutical compositions and kits comprising them are provided and methods for delivering iron to tumors specifically, either intracellularly or extracellularly. As a result, TDI drugs are useful as a sensitizer for radiation therapy, radio-diagnosis, and chemotherapy, and are of interest. In other embodiments, tumor deliverable protein synthesis inhibitors (TDPSI) are provided and can be delivered to tumors, but not normal cells. These TDPSI drugs and pharmaceutical compositions and kits comprising them are useful for their treatment of cancer, either alone or in combination with other active anti-cancer drugs.

### CROSS REFERENCE TO RELATED APPLICATIONS

This application claims the benefit of provisional application, 62/166,837 entitled “Tumor Deliverable Iron and Protein Synthesis Inhibitors as a New Class of Drugs for the Diagnosis and Treatment of Cancer,” filed May 27, 2015, the entire contents of which are incorporated herein.

### BACKGROUND

In the United States, cancer treatment cost $125 billion in 2010 alone. Despite tremendous research and expenses, the progress in the treatment of cancer has been slow. The backbone of cancer treatment today remains cytotoxic drugs that target macromolecules, predominantly DNA, indiscriminatingly. More recently drugs targeting individual proteins, notably kinases, have produced promising results in small subsets of cancers.

Cancer is known to harbor many oncoproteins, mutated tumor suppressor proteins, and mutated DNA sequences due to deletion, translocation, and insertions. Furthermore, many cancers have specific surface markers. These distinct cancer related features, herein referred to as intracellular and surface “cancer markers”, respectively, are obviously potential drug targets. However, there have been only few cases of success by targeting cancer markers in small subsets of cancers. The majority of these cancer markers have been resistant to drug therapy. There are at least two major reasons for this: first, the tremendous heterogeneity of even the same cancer among different patients. For example, overexpression of the c-Myc oncoprotein is considered a driver event for Burkitt lymphoma and many other highly aggressive and chemotherapy resistant lymphomas. However, the exact mechanism of c-Myc overexpression is different among individual patients, and should potentially be treated differently. Secondly, there is a paucity of small molecule inhibitors that can specifically and potently inhibit these cancer markers, demonstrate satisfactory pharmacokinetic features, and are safe to humans.

A platform of precision medicine in the diagnosis and treatment of diverse types of cancer is needed for safe and non toxic therapies.

### SUMMARY

In certain embodiments, tumor deliverable iron (TDI) drugs and pharmaceutical compositions and kits comprising them are provided and methods for delivering iron to tumors specifically, either intracellularly or extracellularly to enhance the detection of cancer by improving the efficacy of radiation and chemotherapy and ultimately diagnosing cancer. As a result, TDI drugs are useful as a sensitizer for radiation therapy, radio-diagnosis, and chemotherapy or a combination of all three.

In other embodiments, tumor deliverable protein synthesis inhibitors (TDPSI) are provided that can be delivered selectively to cancer cells, but not normal cells. These TDPSI drugs and pharmaceutical compositions and kits comprising them are useful for their treatment of cancer, either alone or in combination with other active anti-cancer drugs.

### DETAILED DESCRIPTION

### 1. Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Generally, nomenclatures used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwart, Thomas M. Jessell editors. McGraw-Hill/Appleton & Lange: New York, N. (2000). Unless defined otherwise, all technical and scientific terms used herein have the same meaning as commonly understood by one of ordinary skill in the art.

The term “tumor deliverable iron” (“TDI”) drug as used herein means a compound that utilizes specific monoclonal antibodies or aptamers against tumor antigens. The antibody or aptamer is fused with heme or one of its equivalents or analogs. The general design of the novel class of drugs will be (A: antibody or aptamer against any surface tumor marker)-(B: Any iron containing compound d that can be safely and effectively linked with “A” and delivered to the tumor). In certain embodiments, a linker peptide may be added to connect A and B. This linker should be cleavable inside cancer cells. In certain embodiments, TDI drugs include CD30 TDI and HER2 TDI.

The term “tumor deliverable protein synthesis inhibitor” (“TDPSI) drug as used herein means a compound that utilizes a specific monoclonal antibody or aptamer against tumor antigens, preferably a surface antigen. The antibody (Ab) or aptamer (Ap) is fused via a peptide linker (PL) with PSIs, with the following structure Ab-PL-PSI or Ap-PL-PSI. Ab-PL-PSI or Ap-PL-PSI binds to the respective tumor antigen in cancer cells, and enters the cancer cell through endocytosis. The Ab-PL-PSI or Ap-Pl-PSI will be cleaved inside the cancer cells, wherein the PSI will bind to and inhibit its intracellular target in the protein synthesis machinery, and the Ab or Ap will be recycled to deliver more PSI intracellularly. In certain embodiments, the TDPSI drug is CD30 TDPSI-CHX, CD30 TDPSI-PTM, HER2 TDPSI-CHX, or HER2 TDPSI-PTM.

The term, “active agent” as used herein, collectively refers to TDI drugs or TDPSI drugs as disclosed herein, and biologically active (i.e. therapeutically effective) fragments or derivatives thereof (for delivery of iron or protein synthesis inhibitors respectively and treatment of cancer.

The term “enumerated disease” as used herein means any cancer cell that expresses CD30 or HER2.

As used herein, “administering” an active agent may be performed using any of the various methods or delivery systems well known to those skilled in the art. The administering can also be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally or microinjection.

Unless otherwise specified, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody moiety.

The term “aptamer” as used herein, refers to a molecule from a class of small nucleic acid ligands that are composed of RNA or single-stranded DNA oligonucleotides and have high specificity and affinity for their targets. Similar to antibodies, aptamers interact with their targets by recognizing a specific three-dimensional structure and are thus often referred to as “chemical antibodies.” In contrast to protein antibodies, aptamers offer unique chemical and biological characteristics based on their oligonucleotide properties.

The terms “animal,” “patient,” or “subject” as used herein, mean any animal (e.g., mammals, (including, but not limited to humans, primates, dogs, cattle, cows, horses, kangaroos, pigs, sheep, goats, cats, rabbits, rodents, and transgenic non-human animals), and the like, which are to be the recipient of a particular treatment. Typically, the terms “animal” “subject” and “patient” are used interchangeably herein in reference to a human subject or a rodent. The preferred animal, patient, or subject is a human.

The term “cancer” as used herein, includes the enumerated diseases which are any CD-antigen or HER2 expressing cancers, characterized by the uncontrolled or aberrant growth of aberrant cancer cells. Cancer includes malignant, benign, soft tissue, or solid, and cancers of all stages and grades including pre- and post-metastatic cancers. These cancers can appear on the skin, lips, mouth, lung, head, stomach, prostate, colon, rectum, breast, ovaries, uterus, lymphoma, stomach, throat, urinary tract, reproductive tract, and esophagus.

The term “CD-antigen” as used herein means, a cell surface antigen of a human leukocyte. A cluster of differentiation (cluster of designation or classification determinant) (often abbreviated as CD) is a protocol used for the identification and investigation of cell surface molecules providing targets for immunophenotyping of cells. In terms of physiology, CD molecules can act in numerous ways, often acting as receptors or ligands (the molecule that activates a receptor) important to the cell. A signal cascade is usually initiated, altering the behavior of the cell (see cell signaling). Some CD proteins do not play a role in cell signaling, but have other functions, such as cell adhesion. The proposed surface molecule is assigned a CD number once two specific monoclonal antibodies (mAb) are shown to bind to the molecule. Physiologically, CD antigens do not belong in any particular class of molecules, with their functions ranging from cell surface receptions to adhesion molecules. Although initially used for just human leukocytes, the CD molecule naming convention has now been expanded to cover both other species (e.g. mouse) as well as other cell types. Human CD antigens are currently numbered up to CD363.

The term, “CD19” as used herein, a B-lymphocyte antigen, also known as CD19 (Cluster of Differentiation 19), is a protein that in humans is encoded by the CD19 gene. It is found on the surface of B-cells, a type of white blood cell. An anti-CD19 antibody, or anti-CD19 immunotoxin as used herein is a monoclonal or polyclonal antibody (preferably monoclonal) (targeting CD19) that may be linked to a cytotoxic agent to form an antibody-drug conjugate. It binds to CD19. Anti-CD19 antibodies (e.g., Blinatumomab, Coltuximavravtansine, MOR208, MEDI-551, Denintuzumavmafodotin, Merck patent anti-CD19, Taplitumomabpaptox, XmAb 5871, MDX-1342, and AFM11) are described in the art (see, e.g., Naddafum F., “Anti-CD19 Monoclonal Antibodies: a New Approach to Lymphoma Therapy,” Int J Mol Cell Med. 2015 Summer; 4(3): 143-151.)

The term, “CD20” as used herein, is an activated-glycosylated phosphoprotein expressed on the surface of all B-cells beginning at the pro-B phase (CD45R+, CD117+) and progressively increasing in concentration until maturity. In humans CD20 is encoded by the MS4A1 gene. An anti-CD20 monoclonal antibody, or anti-CD19 immunotoxin as used herein is a monoclonal or polyclonal antibody (preferably monoclonal) (targeting CD20) that may be linked to a cytotoxic agent to form an antibody-drug conjugate. It binds to the CD20 B-cell receptor. Anti-CD20 antibodies (e.g., ofatumumab, obinutuzumab, rituximab/mabthera, ublituximab, and veltuzumab) are described in the art (see, e.g., FDA website describing clinical trials at ClinicalTrials.gov).

The term, “CD22” as used herein, is a receptor protein on the surface of normal B cells and B-cell tumors. An anti-CD22 antibody, or anti-CD22 immunotoxin as used herein is a monoclonal or polyclonal antibody (preferably monoclonal) (targeting CD22) that may be linked to a cytotoxic agent to form an antibody-drug conjugate. It binds to CD22. Anti-CD22 antibodies (e.g., epratuzumab) are described in the art (see, e.g., FDA website describing clinical trials at ClinicalTrials.gov).

The term, “CD30” as used herein, is also known as TNFRSF8, and means a cell membrane protein of the tumor necrosis factor receptor family and tumor marker. CD30 is associated with anaplastic large cell lymphoma. It is expressed in embryonal carcinoma but not in seminoma and is thus a useful marker in distinguishing between these germ cell tumors. CD30 is also expressed on classical Hodgkin Lymphoma Reed-Sternberg cells. An anti-CD30 antibody, or anti-CD30 immunotoxin as used herein is a monoclonal or polyclonal antibody (preferably monoclonal) (targeting CD30) that may be linked to a cytotoxic agent to form an antibody-drug conjugate. It binds to CD30. Anti-CD30 antibodies (e.g., SGN-30) are described in the art (see, e.g., FDA website describing clinical trials at ClinicalTrials.gov).

The term, “CD33” as used herein, or Siglec-3 (sialic acid binding Ig-like lectin 3, SIGLEC3, SIGLEC-3, gp67, p67), is a transmembrane receptor expressed on cells of myeloid lineage. It is usually considered myeloid-specific, but it can also be found on some lymphoid cells. An anti-CD33 antibody, or anti-CD33 immunotoxin as used herein is a monoclonal or polyclonal antibody (preferably monoclonal) (targeting CD33) that may be linked to a cytotoxic agent to form an antibody-drug conjugate. It binds to CD33. Anti-CD33 antibodies (e.g. gemtuzumab ozogamicin) are described in the art (see, e.g., Scott, A., et al., “Monoclonal antibodies in cancer therapy,” Cancer Immun. 2012; 12: 14.)

The term, “CD37” as used herein, or “lleukocyte antigen CD37” is a protein that in humans is encoded by the CD37 gene and is a member of the transmembrane 4 superfamily, also known as the tetraspanin family. An anti-CD37 antibody, or anti-CD37 immunotoxin as used herein is a monoclonal or polyclonal antibody (preferably monoclonal) (targeting CD37) linked to a cytotoxic agent to form an antibody-drug conjugate. It binds to CD37. Anti-CD37 antibodies (e.g., otlertuzumab (formerly known as TRU-016), BI 836826, IMGN529 and (177) Lu-tetulomab) are described in the art (see, e.g., Robak, T., “Anti-CD37 antibodies for chronic lymphocytic leukemia,” Expert Opin Biol Ther. 2014 May; 14(5):651-61. doi: 10.1517/14712598.2014.890182. Epub 2014 Feb. 20.)

The term, “CD38” as used herein, also known as cyclic ADP ribose hydrolase, is a glycoprotein found on the surface of many immune cells (white blood cells), including CD4⁺, CD8⁺, B lymphocytes and natural killer cells. CD38 also functions in cell adhesion, signal transduction and calcium signaling. An anti-CD38 antibody, or anti-CD38 immunotoxin as used herein is a monoclonal or polyclonal antibody (preferably monoclonal) (targeting CD38) that may be linked to a cytotoxic agent to form an antibody-drug conjugate. It binds to CD38. Anti-CD38 antibodies (e.g., Daratumumab, SAR650984, & MOR202) are described in the art (see, e.g., Robert H. Carlson, “Anti-CD38 Monoclonal Antibodies Called Next ‘Blockbuster’ Drug Class for Myeloma,” Oncology Times: 25 Jan. 2015-Volume 37-Issue 2-p 13-15.).

The term, “CD39” as used herein, or, Ectonucleoside triphosphate diphosphohydrolase-1 (gene: ENTPD1; protein: NTPDasel), also known as CD39 (Cluster of Differentiation 39), is a typical cell surface-located enzymes with an extracellularly facing catalytic site. An anti-CD39 antibody, or anti-CD39 immunotoxin as used herein is a monoclonal or polyclonal antibody (preferably monoclonal) (targeting CD39) that may be linked to a cytotoxic agent to form an antibody-drug conjugate. It binds to CD39. Anti-CD39 antibodies (e.g., alemtuzumab, MDX01411, milatuzumab/hLL1, mogamulizumab) are described in the art (see, e.g., Hayes, G., “CD39 is a promising therapeutic antibody target for the treatment of soft tissue sarcoma,” Am J Transl Res. 2015; 7(6): 1181-1188. Published online 2015 Jun. 15.).

The term, “CD52” as used herein, is a protein present on the surface of mature lymphocytes, but not on the stem cells from which these lymphocytes are derived. An anti-CD52 antibody, or anti-CD52 immunotoxin as used herein is a monoclonal or polyclonal antibody (preferably monoclonal) (targeting CD52) that may be linked to a cytotoxic agent to form an antibody-drug conjugate. It binds to CD52. Anti-CD52 antibodies (e.g., alemtuzumab) are described in the art (see, e.g., Pangalis, G., “Campath-1H (anti-CD52) monoclonal antibody therapy in lymphoproliferative disorders,” Med. Oncol. 2001; 18(2): 99-107.

The term, “CD79” as used herein, is a protein composed of CD79a and CD79b components and is expressed almost exclusively on B cells and B-cell neoplasms. An anti-CD79 antibody, or anti-CD79 immunotoxin as used herein is a monoclonal or polyclonal antibody (preferably monoclonal) (targeting CD79) may be linked to a cytotoxic agent to form an antibody-drug conjugate. It binds to CD79. Anti-CD79 antibodies (e.g. HM57,) are described in the art (see, e.g., Zhang, L., “The development of anti-CD79 monoclonal antibodies for treatment of B-cell neoplastic disease,” Ther Immunol. 1995 August; 2(4): 191-202.).

The term “drug loading,” “drug-load,” or “load” as used herein means the ratio of the active drug to the total contents of the dose. A low drug load may cause homogeneity problems. A high drug load may pose flow problems or require large capsules if the compound has a low bulk density. Drug loading may be used as a design parameter to improve drug pharmacokinetics and efficacy. (See, e.g., Cobb, J., “Increase of Drug Load and Potency of a Capsule Formulation by Conversion from a Simple Powder Blend to Roller Compaction,” Metrics Inc.).

The term “EGFR” as used herein, is a cell surface protein that binds to epidermal growth factor. Binding of the protein to a ligand induces receptor dimerization and tyrosine auto phosphorylation and leads to cell proliferation. Mutations in this gene are associated with lung cancer. An anti-EGFR antibody, or anti-EGFR immunotoxin as used herein may be a monoclonal or polyclonal (preferably monoclonal) antibody (targeting EGFR) may be linked to a cytotoxic agent to form an antibody-drug conjugate. It binds to HER-2. Anti-HER2 antibodies (e.g., Cetuximab (Erbitux), Erlotinib, Gefitinib, and Panitumumab (Vectibix) are described in the art (see, e.g., Perez, R., et al., “EGFR-Targeting as a Biological Therapy: Understanding Nimotuzumab's Clinical Effects,” Cancers 2011, 3, 2014-2031; doi: 10.3390/cancers3022014.)

The term “HER2” as used herein, is a member of the human epidermal growth factor receptor (HER/EGFR/ERBB) family. Amplification or overexpression of this oncogene has been shown to play an important role in the development and progression of certain aggressive types of breast cancer. In recent years the protein has become an important biomarker and target of therapy for approximately 30% of breast cancer patients. An anti-HER2 antibody, or anti-HER2 immunotoxin as used herein may be a monoclonal or polyclonal (preferably monoclonal) antibody (targeting HER-2) may be linked to a cytotoxic agent to form an antibody-drug conjugate. It binds to HER-2. Anti-HER2 antibodies (e.g. trastuzumab, pertuzumab) are described in the art (see, e.g., Wong, D., “Recent advances in the development of anti-HER2 antibodies and antibody-drug conjugate,” Ann Transl Med. 2014 December; 2 (12): 122.).

The term, “kit” as used herein, means any manufacture (e.g., a package or container) comprising at least one reagent, e.g., a TDI drug or TDPSI drug for delivery of iron to tumors or PSIs to tumors and ultimately treatment of cancer. In certain embodiments, the manufacture may be promoted, distributed, or sold as a unit for performing the methods of the present invention.

The term, “monoclonal antibodies,” as used herein means identical antibodies produced by a single type of immune cell. In targeted cancer therapy, they are directed against molecules unique to, overexpressed in, or mutated in cancer cells.

The term “sample” as used herein means, any biological specimen obtained from a subject. Samples include, without limitation, a tissue sample (e.g., tumor tissue) such as a biopsy of a tumor or of an area of skin having or suspected of having CD-antigen (e.g., CD-22, CD-30, CD-33, CD-79) or HER2 expressing cancer. A biopsy of cells of a solid tumor or of skin suspected of having CD-antigen or HER2 can be obtained using any technique known in the art.

The term “small molecule inhibitor” as used herein means a drug that interferes with the function of molecules involved in the development and progression of cancer. In certain embodiments, thee small molecule inhibitors interfere with tyrosine kinases.

The term “therapeutically effective amount” as used herein means an amount that achieves the intended therapeutic effect of increasing iron content in tumor tissue or treating CD-antigen (e.g., CD-22, CD-30, CD-33, CD-79) or HER2 expressing cancer in a subject. The full therapeutic effect does not necessarily occur by administration of one dose and may occur only after administration of a series of doses. Thus, a therapeutically effective amount may be administered in one or more administrations per day for successive days.

The term “treating” a disease such as CD-antigen (e.g., CD-22, CD-30, CD-33, CD-79) or HER2 expressing cancer in a patient as used herein, means taking steps to obtain beneficial or desired results, including clinical results. For purposes of this invention, beneficial or desired clinical results include, but are not limited to alleviation or amelioration of one or more symptoms of the CD-antigen (e.g., CD-22, CD-30, CD-33, CD-79) or HER2 expressing cancer diminishing the extent of disease; delaying or slowing disease progression; amelioration and palliation or stabilization of the disease state.

The terms “treat” or “treatment” as used herein, mean both therapeutic treatment and prophylactic or preventative measures, wherein the object is to prevent or slow down (lessen) an undesired physiological change or disorder, such as the development, progression or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having cancer and those with benign tumors or precancerous cells.

As used herein, a “tumor” comprises one or more CD-antigen (e.g., CD-22, CD-30, CD-33, CD-79) or HER2 expressing cancer cells or benign cells or precancerous cells.

### 2. Overview

It has been discovered that TDI and TDPSI drugs, are new avenues for the diagnosis, and treatment of CD-antigen (e.g., CD-22, CD-30, CD-33, CD-79) and HER-2 expressing cancers. Certain embodiments are directed to a novel class of TDI and TDPSI drugs and methods for delivering iron and PSIs respectively using these drugs selectively to tumors, but not to normal cells.

Iron plays an important role in the human body. The unique physical and chemical characteristics of iron make it the ideal carrier of oxygen, as an integral component of hemoglobin. However, too much iron is toxic. Hereditary hemochromatosis is a rare disease, caused by absorption of too much iron from food. The excess iron is stored in vital organs, including the liver, heart and pancreas. The excess iron in the cells of these organs causes build-up of reactive oxygen species (ROS), leading to cell death and chronic inflammation. As a result, patients with hemochromatosis die from cancer, heart arrhythmias and cirrhosis. Traditionally, iron deposition in these vital organs is diagnosed by risky biopsy procedures, typically at the late stage. More recently, MRI has been found to be highly specific and sensitive to detect iron deposition in these organs, leading to early diagnosis and intervention and improvement in patient outcome. Two important lessons about iron can be learned from the rare disease of hemochromatosis: first, that increase of intracellular iron can cause ROS build-up and cell death, secondly that the iron level of any tissue can change its magnetic field and how it appears on MRI scan. The scientific rationale of the current invention is that if iron can be delivered specifically to cancer cells, cancer cells will build up ROS and change its magnetic field. These cancer cells with iron deposition will become more sensitive to chemo therapy and radiation therapy because of their high level of ROS. Furthermore, these cancer cells can be detected more easily by MRI due to their high levels of iron and magnetic field.

The hallmark of cancer cells is their relentless and dysregulated growth, which requires robust protein synthesis. Oncoproteins are required to further stimulate the transcription and translational programs. Moreover, oncoproteins, including Myc, are involved in the resistance of cancer cells to chemotherapy. Many oncoproteins such as Myc are well known to have very short half-life, therefore are themselves exquisitely sensitive to inhibition of protein synthesis. The overall scientific rationale of the current invention is that targeting dysregulated protein synthesis will lead to down regulation of oncoproteins, mitigation of chemoresistance, and death of cancer cells.

A number of compounds have been found to specifically and potently inhibit various stages of protein synthesis and cause death of cancer cells. These potent and specific protein synthesis inhibitors (PSIs) include recently discovered silvestrol, hippuristanol, and their analogs. Interestingly, more potent PSIs have existed for more than 2 decades, including, for example, cycloheximide (CHX) and pateamine (PTM). Unfortunately, neither the newer nor older potent PSIs have entered clinical practice. The failure of specific and potent PSIs to enter the clinic is due to their lack of selectivity for cancer cells and unfavorable chemical and pharmacological features, which altogether prevent their successful use in human. In contrast, homoharringtonine (Synribo) was approved in 2012 for chronic myeloid leukemia. While the exact mechanism of homoharringtonine is pleiotropic and remains to be elucidated, the drug is able to inhibit the elongation step of protein synthesis with moderate potency, attesting to the promise of more potent PSIs.

### 3. Embodiments

New classes of anti-cancer drugs are necessary to overcome drug resistance, expense, and unwanted side effects. Drugs that recognize intracellular cancer-specific targets, but do not recognize any extracellular, or cell surface, cancer-specific markers such as kinase inhibitors and proteasome inhibitors are available. Drugs that recognize cell surface cancer-specific markers, but do not target intracellular cancer-specific targets include monoclonal antibodies (e.g., Rituximab, Herceptin, and Pembrolizumab) have proven to be expensive. The majority of cancer patients do not benefit from these new drugs, which tend to be more effective for niche, or rare, cancers. Antibody-Drug Conjugate (ADC) drugs where a monoclonal antibody against a cancer-specific surface marker is linked to a peptide linker which is linked to a load/toxin are in various stages of clinical development but have serious limitations. For example, brentuximab vedotin for Hodgkin lymphoma and anaplastic large cells lymphoma and ado-trastuzumab emtansine for breast cancer are approved. But these ADC drugs rely solely on the monoclonal antibody binding to the cell surface cancer-specific marker to achieve specificity against cancer cells. This is insufficient in that there is no selective recognition of cancer-specific intracellular targets. Instead, current loads target intracellular structures that are indistinguishable from normal cells, using highly potent toxins, including MMAE and DM4, which target the microtubules, and calicheamicin, which targets DNA.

Currently approved ADC drugs are associated with significant adverse effects, including dose limiting neurologic toxicity due to their loads. Cancer cells develop resistance to ADC drugs. Despite an impressive complete response rate of over 75% by brentuximab in the rare lymphoma ALCL, the lymphoma will inevitably relapse with a few months to a few years. Current ADC technology fails to address the complex biology that drives cancer development and accounts for the malignant phenotype, including relentless growth and resistance to chemotherapy.

The present invention overcomes these failures by creating personalized cancer care. Cancer is increasingly defined by alterations at the molecular level. Whole genomic sequencing of individual tumors allows for discovery of different mutations in different patients with even the same type of cancer. Genetic and epigenetic abnormalities of oncogenes and tumor suppressor genes are known to cause the majority of the malignant phenotypes of cancer (e.g., K-ras, c-Myc, p53, Bcl-2, Bcl-6, NF-kB, and NOTCH). Some of the most important oncoproteins (K-ras, c-Myc) remain undruggable, despite their molecular functions having been well characterized. More effective therapies are needed that can use drugs to target distinct molecular alterations in cancer of different patients.

### A. Methods of Nucleotide Targeting and Protein Targeting Aided by Surface Antigen Binding

A platform for targeting nucleotides and proteins aided by surface antigen binding is provided for the treatment of various cancers (e.g., B-cell lymphoma, leukemia, HER-2 positive breast cancer, EGFR positive pancreatic, head, neck, and other cancers) and PSA positive prostate cancer). Herein, this platform is described that may form the foundation of precision medicine in the treatment of certain types of cancer.

In certain embodiments, the platform “AA-LINK-NUPRO” has the following backbone:

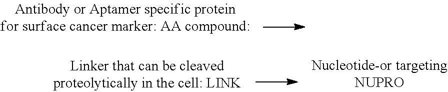

Examples of potential drugs that can be made from this platform include, but are not limited to, the following compounds listed in Table 1.

TABLE 1
AA-LINK-NUPRO Platforms.

For Treatment of B-cell Lymphoma
(Anti-CD20 Antibody, whole or a portion of)-LINK-(microRNA to MYC gene)
(Anti-CD20 Antibody, whole or a portion of)-LINK-(microRNA to BCL2 gene
(Anti-CD20 Antibody, whole or a portion of)-LINK-(microRNA to BCL6 gene)
(Anti-CD19 Antibody, whole or a portion of)-LINK-(microRNA to MYC gene)
(Anti-CD19 Antibody, whole or a portion of)-LINK-(microRNA to BCL2 gene)
(Anti-CD19 Antibody, whole or a portion of)-LINK-(microRNA to BCL6 gene)
(Anti-CD19 Antibody, whole or a portion of)-LINK-(Aptamer that binds and inactivates Myc
protein)
(Anti-CD19 Antibody, whole or a portion of)-LINK-(Aptamer that binds and inactivates Bcl2
protein)
(Anti-CD19 Antibody, whole or a portion of)-LINK-(Aptamer that binds and inactivates Bcl6
protein)
(Anti-CD19 Antibody, whole or a portion of)-LINK-(nucleotide-invading-peptide that binds to
translocated MYC gene specifically)
For Treatment of Poor Risk Acute Leukemia
(Anti-CD34 Antibody, whole or a portion of)-LINK-(microRNA to translocated FLT3-ITD gene)
(Anti-CD34 Antibody, whole or a portion of)-LINK-(Aptamer that binds and inactivates
FLT3-ITD protein)
(Anti-CD34 Antibody, whole or a portion of)-LINK-(nucleotide-invading-peptide that binds to
translocated FLT3-ITD gene)
For Treatment of HER2 Positive Breast Cancer
(Anti-Her2 Antibody, whole or a portion of)-LINK-(Aptamer that binds and inactivates Her2
protein)
For Treatment of EGRF Positive Pancreatic, Head, Neck, and Other Cancers
(Anti-EGFR Antibody, whole or a portion of)-LINK-(Aptamer that binds and inactivates Ras
protein)
(Anti-EGFR Antibody, whole or a portion of)-LINK-(Aptamer that binds and inactivates
mutant p53 protein)
For Treatment of PSA Positive Prostate Cancer
(Anti-PSA Antibody, whole or a portion of)-LINK-(nucleotide-invading-peptide that binds to
recurrent genetic mutations identified through whole genome sequencing of tumor tissues)

Novel platforms of anti-cancer drugs powered by surface recognition and inhibition of cancer-specific intracellular proteins or genes (SPRIIPG) are provided in certain embodiments.

For example, using this platform, it is possible to target the c-Myc oncoprotein in lymphoma using SPRIIPG.

In Module 1 (SR), cancer-specific surface markers are recognized. In certain embodiments, monoclonal antibodies that are specifically designed or commercial available are used for development of SRIIPG prototypes. Monoclonal antibodies are developed by injecting animals (usually mice) with purified target proteins, causing the animals to make many different types of antibodies against the target. These antibodies are then tested to find the ones that bind best to the target without binding to nontarget proteins. Before monoclonal antibodies are used in humans, they are “humanized” by replacing as much of the mouse antibody molecule as possible with corresponding portions of human antibodies. Humanizing is necessary to prevent the human immune system from recognizing the monoclonal antibody as “foreign” and destroying it before it has a chance to bind to its target protein. Humanization is not an issue for small-molecule compounds because they are not typically recognized by the body as foreign. It is commonly believed that antibodies are too large (˜150 kDa) to access the intracellular compartment. Therefore, therapeutic antibodies have been traditionally used to target cell surface receptors or soluble proteins in the circulation, leaving a large intracellular treasure of potential cancer-specific targets untapped.

In Module 1, certain embodiments include aptamers that are also specifically designed (see, Xiaohua Ni, et al., “Nucleic acid aptamers: clinical applications and promising new horizons,” Curr Med Chem. 2011; 18(27): 4206-4214) and available commercially and offer a cheaper and potentially safer SR than monoclonal antibodies for recognition of cell surface cancer-specific markers. Aptamers as used herein, are a class of small nucleic acid ligands that are composed of RNA or single-stranded DNA oligonucleotides and have high specificity and affinity for their targets. Similar to antibodies, aptamers interact with their targets by recognizing a specific three-dimensional structure and are thus termed “chemical antibodies.” In contrast to protein antibodies, aptamers offer unique chemical and biological characteristics based on their oligonucleotide properties. Hence, they are more suitable for the development of novel clinical applications. Aptamers, composed of single-stranded DNA or RNA oligonucleotides that interact with target molecules through a specific three-dimensional structure, are selected from pools of combinatorial oligonucleotide libraries. With their high specificity and affinity for target proteins, ease of synthesis and modification, and low immunogenicity and toxicity, aptamers are considered to be attractive molecules for development as anticancer therapeutics. Two aptamers—one targeting nucleolin and a second targeting CXCL12—are currently undergoing clinical trials for treating cancer patients, and many more are under study. (See, Ji Won Lee, “Therapeutic aptamers: developmental potential as anticancer drugs,” BMB Rep. 2015 April; 48(4): 234-237. doi: 10.5483/BMBRep.2015.48.4.277. For example, AS1411 targets nucleolin, a protein over-expressed in a variety of tumors. This aptamer is currently being evaluated as a potential treatment option in solid tumors and acute myeloid leukemia. Therapeutic aptamers are known in the art and are not limited to those included in Schmidt, K S, Borkowski, S, Kurreck, J, Stephens, A W, Bald, R, Hecht, M et al. (2004). Application of locked nucleic acids to improve aptamer in vivo stability and targeting function. Nucleic Acids Res 32: 5757-5765 and in Table 2.

TABLE 2
Therapeutic Aptamers.

| Aptamer | Molecular target | Sponsor | Medical Indications | Current status |
|---|---|---|---|---|
| ARC1779 | Activated von Willebrand | Archamix Corporation | Purpura; Thrombotic Thrombocytopenic; | Phase 2 completed |
| | Factor (vWF) | | Von Willebrand Disease Type-2b | |
| ARC1905 | Complement factor C5 | Ophthotech Corporation | Age-Related Macular Degeneration | Phase 1 completed |
| ARC19499 | Tissue Factor Pathway | Baxter Healthcare | Hemophilia | Phase 1 terminated |
| | Inhibitor (TFPI) | Corporation | | |
| AS1411 | Nucleolin | Antisoma Research | Leukemia, Myelold | Phase 2 completed |
| | | | Metastatic Renal Cell Carcinoma | Phase 2 status is |
| | | | | unknown |
| E10030 | Platelet-derived growth | Ophthotech Coproration | Age-Related Macular Degeneration | Phase 3 recruiting |
| | factor (PDGF) | | | participants |
| NOX-E36 | Monocyte Chemoattractant | NOXXON Pharma AG | Type 2 Diabetes Mellitus: Albuminuria | Phase 2 completed |
| | Protein-1 (MCP-1) | | | |
| NOX-A12 | Stromal Cell-Derived | NOXXON Pharma AG | Multiple Myeloma; Chronic | Phase 2 recruiting |
| | Factor-1 | | Lymphocytic Leukemia | participants |
| NOX-H94 | Hepcidin | NOXXON Pharma AG | Anemia of Chronic Disease | Phase 2 completed |
| NU172 | Thrombin (Factor IIa) | ARCA Biopharma | Heart Disease | Phase 2 status is |
| | | | | unknown |
| REG1 | Coagulation factor IX | Regado Biosciences | Coronary Artery Disease | Phase 3 recruiting |
| | | | | participants |

See also, G. Mayer, “The chemical biology of aptamersm,” Angew Chem Int Ed Engl. 2009; 48(15):2672-89. doi: 10.1002/anie.200804643.

One of the biggest advantages of use of aptamers in Module 1, compared with antibodies, is the ease with which they can be modified chemically while retaining target specificity. Accordingly, there have been numerous efforts to combine the high target-specificity of aptamers with other anticancer modalities to provide targeted delivery of a variety of drg payloads. In these applications, aptamers that target cancer-specific membrane proteins mediate precise delivery of anti-cancer agents, such as nanoparticles, siRNA/miRNA, or cytotoxic drugs, to tumor cells. After binding target membrane proteins, aptamers are internalized into the cell together with their drg payload. Ultimately, the drgs are then released from the target molecules and exert their anticancer functions by damaging DNA or inhibiting microtubule polymerization. In one example of a nanoparticle designed for prostate cancer therapy, an RNA aptamer targeting prostate-specific membrane antigen (PSMA) was conjugated with a PLA (polylactide)-PEG or PLGA (polylactide-co-glycolide)-PEG nanoparticle encapsulating docetaxel. In another example, paclitaxel-containing PLGA conjugated with an aptamer against mucin-1 (MUC1) was used to target MUC 1-expressing cancer cells. siRNA/miRNA payloads have also been conjugated directly to aptamers. For example, chimeric complexes of Plk1 or Bcl2 siRNA-PSMA aptamers and doxorubicin-PSMA aptamers have been developed for inhibiting PSMA-expressing prostate cancers. Aptamer drg conjugates (ApDCs), which are conceptually similar to antibody-drug conjugates (ADCs), are also promising technologies for targeted cancer therapy because they can enhance therapeutic efficacy while reducing associated toxicities. Several potential problems with the ADCs approach remain to be resolved, such as undefined antibody-toxin ratios due to heterogeneous drug conjugation, a tendency to aggregate during synthesis, poor pharmacokinetics, and loss of immune reactivity. However, the beneficial properties of aptamers, such as accurate site conjugation and high solubility (>150 mg/ml), may ultimately surmount these potential issues. Accordingly, when referring to an antibody specific to a target antigen, such as in reference to module1 or specific conjugate embodiments described herein, those skilled in the art will appreciate that the antibody may be substituted with an aptamer specific to the target antigen.

In Module 2, a peptide linker is provided. Control of structural flexibility is essential for the proper functioning of a large number of proteins and multiprotein complexes. At the residue level, such flexibility occurs due to local relaxation of peptide bond angles whose cumulative effect may result in large changes in the secondary, tertiary or quaternary structures of protein molecules. Such flexibility, and its absence, most often depends on the nature of interdomain linkages formed by oligopeptides. Both flexible and relatively rigid peptide linkers are found in many multidomain proteins. Linkers are thought to control favorable and unfavorable interactions between adjacent domains by means of variable softness furnished by their primary sequence. Large-scale structural heterogeneity of multidomain proteins and their complexes, facilitated by soft peptide linkers, is now seen as the norm rather than the exception. Biophysical discoveries as well as computational algorithms and databases have reshaped our understanding of the often spectacular biomolecular dynamics enabled by soft linkers. Absence of such motion, as in so-called molecular rulers, also has desirable functional effects in protein architecture. We review here the historic discovery and current understanding of the nature of domains and their linkers from a structural, computational, and biophysical point of view. A number of emerging applications, based on the current understanding of the structural properties of peptides, are presented in the context of domain fusion of synthetic multifunctional chimeric proteins. (See, W. Wriggers, et al., “Control of protein functional dynamics by peptide linkers,” Biopolymers. 2005; 80(6):736-46.) Empirical linkers designed by researchers are generally classified into 3 categories according to their structures: flexible linkers, rigid linkers, and in vivo cleavable linkers. Besides the basic role in linking the functional domains together (as in flexible and rigid linkers) or releasing the free functional domain in vivo (as in in vivo cleavable linkers), linkers may offer many other advantages for the production of fusion proteins, such as improving biological activity, increasing expression yield, and achieving desirable pharmacokinetic profiles. Examples for their design and application can also be found in Chen, X., et al., “Fusion protein linkers: property, design and functionality,” Adv Drug Deliv Rev. 2013 October; 65 (10): 1357-69). Examples of linkers that may be utilized in accord with the teachings herein include, but are not limited, to those provide in Table 5 below.

In Module 3 (IIPG), in certain embodiments, cancer-specific intracellular targets are inhibited with small molecule inhibitors. Small molecule inhibitors are available and known in the art (see, e.g., Hoelder, S., “Discovery of small molecule cancer drugs: Successes, challenges and opportunities,” Molecular Oncology Volume 6, Issue 2, April 2012, Pages 155-176, Edited By John Mendelsohn, Ulrik Ringborg and Richard Schilsky) and potently target intracellular cancer-specific targets, e.g., K-ras. In certain embodiments, these inhibitors are used as the loads. (e.g., supply of loads is potentially unlimited using microRNA for targeting). Specificity against cancer is ensured first by the recognition of cell surface cancer-specific markers using the SPIIPG platform. Through the specific interaction of the inhibitor (e.g., chemical compound, microRNA), and its intracellular cancer-specific target allow for specificity against cancer. Potentially much fewer adverse effects since highly potent toxins for non-specific intracellular targets are not used as the loads. In using the present invention selective delivery to cancer cells using drugs that target the complex biology underlying cancer's growth and resistance to chemotherapy.

However, a concern known as the ‘druggability gap’ exists. Many targets with very promising disease linkage, such as mutated RAS proteins or transcription factors like c-MYC or hypoxia-inducible factor (HIF), are currently regarded as technically undruggable—or at the very least as extremely challenging to target by medicinal chemists using small molecules (Verdine and Walensky, 2007). Even in the case of more druggable proteins it takes several years to identify a drug candidate that satisfies the stringent requirement for clinical development (Paul et al., 2010).

Small-molecule compounds are typically developed for targets that are located inside the cell because such agents are able to enter cells relatively easily. Candidate small molecules are usually identified in what are known as “high-throughput screens,” in which the effects of thousands of test compounds on a specific target protein are examined. Compounds that affect the target (sometimes called “lead compounds”) are then chemically modified to produce numerous closely related versions of the lead compound. These related compounds are then tested to determine which are most effective and have the fewest effects on nontarget molecules.

In certain embodiments, small molecule inhibitors typically interrupt cellular processes by interfering with the intracellular signaling of tyrosine kinases (i.e., enzymes that transfer phosphate groups from adenosine triphosphate to tyrosine amino acid residues in proteins). Tyrosine kinase signaling initiates a molecular cascade that can lead to cell growth, proliferation, migration, and angiogenesis in normal and malignant tissues. EGFR, HER2/neu, and VEGF receptors are tyrosine kinases. They are usually administered orally rather than intravenously. They are chemically manufactured, a process that is often much less expensive than the bioengineering required for monoclonal antibodies. They achieve less specific targeting than do monoclonal antibodies, as evident in the multitargeting nature of the kinase inhibitors imatinib (Gleevec), dasatinib (Sprycel), sorafenib (Nexavar), and sunitinib (Sutent). Unlike monoclonal antibodies, most small molecule inhibitors are metabolized by cytochrome P450 enzymes, which may result in interactions with such medications as macrolide antibiotics, azole antifungals, certain anticonvulsants, protease inhibitors, warfarin, and St. John's wort. Whereas monoclonal antibodies have half-lives ranging from days to weeks (and are therefore usually administered once every one to four weeks), most small molecule inhibitors have half-lives of only hours and require daily dosing. Examples of small molecule inhibitors are described in the art and include, but are not limited to those set forth in Table 3.

TABLE 3
Small Molecule Inhibitors for Cancer Treatment

| | | FDA- | TOXICITIES, SIDE | |
|---|---|---|---|---|
| | | APPROVED | EFFECTS, AND | |
| DRUG | TARGET | INDICATIONS | PRECAUTIONS | MONITORING |
| Bortezomib | 26S | Multiple | Peripheral | Signs and |
| (Velcade) | proteasome | myeloma, mantle | neuropathy; | symptoms of |
| | | cell lymphoma (a | myelosuppression; | peripheral |
| | | subtype of non- | rash; constipation; | neuropathy; CBC |
| | | Hodgkin's | diarrhea; edema; | |
| | | lymphoma) | nausea and | |
| | | | vomiting | |
| Dasatinib | BCR-ABL, | Chronic myeloid | Rash; diarrhea; | CBC; ECG; liver |
| (Sprycel) | SRC family, | leukemia, acute | pleural effusion; | chemistries; |
| | c-KIT, | lymphocytic | fluid retention; | weight; signs and |
| | PDGFR | leukemia | mucositis; | symptoms of fluid |
| | | | myelosuppression; | retention |
| | | | QT interval | |
| | | | prolongation | |
| Erlotinib | EGFR | Non-small cell | Acneiform rash; | Liver chemistries; |
| (Tarceva) | | lung cancer, | diarrhea; loss of | signs of |
| | | pancreatic | appetite; nausea | inflammatory or |
| | | cancer | and vomiting; | infectious |
| | | | fatigue; | sequelae in |
| | | | conjunctivitis; | patients with |
| | | | elevated liver | dermatologic |
| | | | chemistries | toxicity |
| Gefitinib | EGFR | Non-small cell | Acneiform rash; | Liver chemistries; |
| (Iressa) | | lung cancer | diarrhea; loss of | signs of |
| | | | appetite; interstitial | inflammatory or |
| | | | lung disease (rare); | infectious |
| | | | elevated liver | sequelae in |
| | | | chemistries | patients with |
| | | | | dermatologic |
| | | | | toxicity |
| Imatinib | BCR-ABL, | Acute | Rash; weight gain; | CBC; liver |
| (Gleevec) | c-KIT, | lymphocytic | edema; pleural | chemistries; |
| | PDGFR | leukemia, | effusion; cardiac | weight; signs and |
| | | chronic myeloid | toxicity (depression | symptoms of fluid |
| | | leukemia, | of LVEF); nausea | retention |
| | | gastrointestinal | and vomiting; | |
| | | stromal tumor, | arthralgias and | |
| | | hypereosinophilic | myalgias; | |
| | | syndrome, | myelosuppression | |
| | | systemic | | |
| | | mastocytosis | | |
| Lapatinib | HER2/neu, | Breast cancer | Cardiac toxicity | LVEF; ECG; |
| (Tykerb) | EGFR | with HER2/neu | (depression of | electrolyte levels; |
| | | overexpression | LVEF; QT | liver chemistries |
| | | | prolongation); | |
| | | | acneiform rash; | |
| | | | palmar-plantar | |
| | | | erythrodysesthesia | |
| | | | (hand-foot | |
| | | | syndrome); | |
| | | | diarrhea; nausea | |
| | | | and vomiting; | |
| | | | elevated liver | |
| | | | chemistries | |
| Sorafenib | BRAF, | Renal cell | Hypertension; | Blood pressure; |
| (Nexavar) | VEGFR, | cancer, | alopecia; bleeding; | dermatologic |
| | EGFR, | hepatocellular | rash; palmar- | toxicity (including |
| | PDGFR | carcinoma | plantar | palmar-plantar |
| | | | erythrodysesthesia | erythrodysesthesia |
| | | | (hand-foot | [hand-foot |
| | | | syndrome); | syndrome]); |
| | | | hypophosphatemia; | amylase, lipase, |
| | | | diarrhea; nausea | and phosphate |
| | | | and vomiting; | levels; CBC |
| | | | elevated amylase | |
| | | | and lipase levels; | |
| | | | myelosuppression; | |
| | | | wound-healing | |
| | | | complications | |
| | | | Discontinue | |
| | | | treatment | |
| | | | temporarily for | |
| | | | surgical procedures | |
| Sunitinib | VEGFR, | Renal cell | Nausea and | Adrenal function in |
| (Sutent) | PDGFR, c- | cancer, | vomiting; yellow | patients with |
| | KIT, FLT3 | gastrointestinal | discoloration of | trauma or severe |
| | | stromal tumor | skin; | infection, or in |
| | | | hypothyroidism; | those undergoing |
| | | | depression of | surgery; blood |
| | | | LVEF; adrenal | pressure; ECG; |
| | | | function | LVEF; CBC; |
| | | | abnormalities; | electrolyte levels |
| | | | diarrhea; | (magnesium and |
| | | | myelosuppression; | potassium); |
| | | | mucositis; elevated | phosphate levels; |
| | | | lipase and | signs and |
| | | | creatinine levels; | symptoms of |
| | | | elevated liver | pancreatitis; |
| | | | chemistries; | thyroid function |
| | | | increased uric acid | tests |
| | | | levels | |

In certain embodiments, small molecule inhibitors are administered orally except bortezomib, which is administered intravenously. Most small molecule inhibitors undergo metabolism by cytochrome P450 enzymes and are therefore subject to multiple potential interactions.

### B. Dosage

Determining optimal dosing is one challenge. Where the compounds of the invention are administered in conjunction with other therapies, dosages of the co-administered compounds will of course vary depending on the type of co-drug employed, on the specific drug employed, on the condition being treated and so forth.

Clinical trials of traditional chemotherapeutic drugs generally determine toxicity through the degree of myelosuppression. Targeted therapies that include monoclonal antibodies or small molecule inhibitors, however, often do not cause significant hematologic toxicity. Assessment of treatment effectiveness also may require a paradigm shift. When traditional chemotherapy is effective, reduction in tumor volume is anticipated on serial radiographic studies. In contrast, some targeted therapies may impart a clinical benefit by stabilizing tumors, rather than shrinking them.

To determine the dosing and effectiveness of targeted therapies, cancer researchers increasingly are turning to pharmacodynamic end points, such as tumor metabolic activity on positron emission tomography scans, levels of circulating tumor and endothelial cells, and serial levels of target molecules in tumor tissue. These studies add complexity and cost to clinical research. In addition, repeat biopsies of tumor tissue may be inconvenient for patients and unacceptable to institutional review boards. Although these studies may initially increase the time and expense of therapy, they may improve its long-term cost-effectiveness by identifying the subset of patients most likely to benefit from specific drugs.

Dosing strategies for anticancer drugs are known in the art and can be found in Physicians Desk Reference and other references such as “Dosing strategies for anticancer drugs: the good, the bad and body-surface area,” A Felici, J Verweij, A Sparreboom—European Journal of Cancer, 2002. For example, AZD2014 has been administered orally to solid tumor cancer patients in single doses up to 100 mg and multiple doses up to 100 mg twice daily (BID). In other examples, patients receiving oral GSK458 once or twice daily in a dose escalation design to define the maximally tolerated dose (MTD). Expansion cohorts evaluated pharmacodynamics (PD), pharmacokinetics (PK), and clinical activity in histologically- and molecularly-defined cohorts. 170 patients received doses ranging from 0.1 to 3 mg once or twice daily. See, Munster P., et al., “First-in-Human Phase I Study of GSK2126458, an Oral Pan-Class I Phosphatidylinositol-3-Kinase Inhibitor, in Patients with Advanced Solid Tumor Malignancies,” Clin Cancer Res. 2016 Apr. 15; 22(8):1932-9. doi: 10.1158/1078-0432.CCR-15-1665. Epub 2015 Nov. 24.

Dosing is dependent on severity and responsiveness of the condition to be treated, with course of treatment lasting from several days to several months or until a reduction in HIV viral titre (routinely measured by Western blot, ELISA, RT-PCR, or RNA (Northern) blot) is effected or a diminution of disease state is achieved. Optimal dosing schedules are easily calculated from measurements of drug accumulation in the body. Persons of ordinary skill can easily determine optimum dosages, dosing methodologies, and repetition rates. Therapeutically or prophylactically effective amounts (dosages) may vary depending on the relative potency of individual compositions, and can generally be routinely calculated based on molecular weight and EC50s in in vitro and/or animal studies. For example, given the molecular weight of drug compound (derived from sequence and chemical structure) and an experimentally derived effective dose such as an IC₅₀, for example, a dose in mg/kg is routinely calculated. In general, dosage is from 0.001 μg to 100 g and may be administered once or several times daily, weekly, monthly, yearly, or even every decade.

### C. TDI Drugs and Methods of Delivery of Iron to Tumors for Enhancing Detection of Cancer

Current radiological diagnosis, including mainly MRI, CT, and PET scans, cannot effectively distinguish tumorous masses from inflammatory masses. Furthermore, radiation therapies today cannot differentiate tumor tissues from surrounding normal tissues, and are associated with significant toxicities such as radiation burn, tissue/organ damages, bone marrow injury and failure. Cancer cells also develop resistance to radiation. TDI drugs markedly enhance the sensitivity of cancer including tumors to radiation, without affecting normal tissues. As a result, TDI drugs can be used in conjunction with MRI and CT scans, and can substantially improve the ability to diagnose cancer through radiological studies. The TDI drugs as a class can deliver iron to tumors specifically, either intracellularly or extracellularly, to enhance the detection of cancer and improve the efficacy of radiation and chemotherapy. Therefore, TDI drugs are useful as a sensitizer for radiation therapy, radio-diagnosis, and chemotherapy, or a combination of all three.

In certain embodiments, an antibody (Ab) or aptamer (Ap) in Module 1 will be fused with an iron containing compound in Module 3 such as Cytochrome C (structure below) which directly stimulates programmed cell death (apoptosis) in cancer cells and improves the efficacy of radio- and chemo-therapy.

In certain embodiments, an antibody (Ab) or aptamer (Ap) will be fused with an iron containing compound such as heme (structure below) or one of its equivalents or analogs. Heme is a naturally occurring compound normally found in the human body. Heme has an iron molecule in its core, and is a component of hemoglobin. Iron increases DNA damage and modifies the magnetic field. It can be used to enhance the sensitivity of MRI, and improve the efficacy of radio- and chemo-therapy.

In certain embodiments, an antibody (Ab) or aptamer (Ap) will be fused with an iron containing compound such as Monomethyl auristatin E (structure below). Monomethyl auristatin E (MMAE) is used in the approved drug brentuximab vedotin and a number of other antibody drug conjugates (ADCs) in clinical trials.

The general design of this novel class of drugs known as TDI will be the following: (A: antibody or aptamer against any cell surface tumor marker)-(B: Any iron containing compound that can be safely and effectively linked with “A” and delivered to the tumor). Further, a linker peptide maybe added to connect A and B. The linker should be cleavable inside the cancer cells. This class of drgs will be defined as Tumor Deliverable Iron (TDI).

In certain embodiments, the specific structures of two candidate TDI drugs from this class will be the following:
CD22 TDI: (A: anti-CD22 monoclonal antibody)-(B: Heme or its analog). It will be used for CD22 expression blood and solid tumors.
CD30 TDI: (A: anti-CD30 monoclonal antibody)-(B: Heme or its analog). It will be used for CD30 expression blood and solid tumors, primarily lymphoma.
CD33 TDI: (A: anti-CD33 monoclonal antibody)-(B: Heme or its analog). It will be used for CD33 expression blood and solid tumors.
CD79 TDI: (A: anti-CD79 monoclonal antibody)-(B: Heme or its analog). It will be used for CD30 expression blood and solid tumors and
HER2 TDI: (A: anti-HER2 monoclonal antibody)-(B: Heme or its analog). It will be used for HER2 expressing tumors, primarily HER2 positive breast cancer.

The TDI drugs as a class can deliver iron to tumors specifically, either intracellularly or extracellularly. As a result, TDI drugs are useful as a sensitizer for radiation therapy, radio-diagnosis, and chemotherapy.

### D. TDPSI Drugs

TDPSIs in certain embodiments may include specific and potent PSIs such as CHX (cycloheximide) and PTM (pateamine). The targets of CHX and PRM are micromolecules involved in protein synthesis, which are inherently different between cancer and normal cells. In certain embodiments, an antibody (Ab) (e.g., anti-CD19, anti-CD20, anti-CD30, anti-CD33, anti-CD37, anti-CD79, anti-HER2, and anti-EGFR) will be fused via a peptide linker (PL) with PSIs, with the following structure Ab-PL-PSI. Ab-PL-PSI will bind to the tumor cell surface antigen in cancer cells, and enter the cancer cells through endocytosis. The Ab-PL-PSI will be cleaved inside the cancer cells, where the PSI will bind to and inhibit its intracellular target in the protein synthesis machinery, and the Ab will be recycled to deliver more PSI intracellularly.

In certain embodiments, the specific structures of candidate TDPSI drugs include the following:

CD30 TDPSI-CHX: Anti-CD30 monoclonal antibody—PL-cycloheximide.

It will be used for CD30 expressing tumors, primarily CD30 positive lymphoma in certain embodiments.

CD30 TDPSI-PTM: Anti-CD30 monoclonal antibody—PL-pateamine.

It will be used for CD30 expressing tumors, primarily CD30 positive lymphoma in certain embodiments.

CD30 TDPSI-PTM: Anti-CD30 monoclonal antibody—PL-silvestrol.

CD30 TDPSI-PTM: Anti-CD30 monoclonal antibody—PL-hippuristanol.

CD20 TDPSI-PTM: Anti-CD20 monoclonal antibody—PL-cycloheximide

CD20 TDPSI-PTM: Anti-CD20 monoclonal antibody—PL-pateamine

CD20 TDPSI-PTM: Anti-CD20 monoclonal antibody—PL-silvestrol

CD20 TDPSI-PTM: Anti-CD20 monoclonal antibody—PL-hippuristanol

HER2 TDPSI-CHX: Anti-HER2 monoclonal antibody—PL-cycloheximide.

It will be used for HER2 expressing tumors, primarily HER2 positive breast cancer in certain embodiments.

HER2 TDPSI-PTM: Anti-HER2 monoclonal antibody—PL-pateamine.

It will be used for HER2 expressing tumors, primarily HER2 positive breast cancer in certain embodiments.

HER2 TDPSI-PTM: Anti-HER2 monoclonal antibody—PL-silvestrol.

It will be used for HER2 expressing tumors, primarily HER2 positive breast cancer in certain embodiments.

HER2 TDPSI-PTM: Anti-HER2 monoclonal antibody—PL-hippuristanol.

It will be used for HER2 expressing tumors, primarily HER2 positive breast cancer in certain embodiments.

The TDPSI drugs as a class deliver potent and specific PSIs to tumors, but not to normal cells. As a result, TDPSI drugs are useful for the treatment of cancer, either alone or in combination with other active anti-cancer drugs. Because TDPSIs may preferentially inhibit the synthesis of oncoproteins that are involved in resistance to chemotherapy, TDPSI drugs may be used at very low and safe doses with the primary goal of turning off those oncoproteins involved in chemoresistance. The cancer cells will then become highly sensitive to existing chemotherapeutic agents. In the latter scenario, TDPSI will be used as a priming agent, or adjuvant therapy, to overcome or avoid resistance to chemotherapy.

### E. Methods of Delivering TDPSI Drugs to Tumors and Treatment of Cancer

In certain embodiments, a method is provided to deliver potent and specific PSIs to cancer cells, thereby circumventing potential toxicities and enhancing the introduction of this class of drugs into clinical practice. The method will utilize specific monoclonal antibodies against tumor antigens. The TDPSI drugs as a class can deliver potent and specific PSIs to tumors, but not to normal cells. As a result, TDPSI drugs are useful for the treatment of CD-antigen or HER2 expressing cancers, either alone or in combination with other active anti-cancer drugs known in the art.

Because TDPSIs may preferentially inhibit the synthesis of oncoproteins that are involved in resistance to chemotherapy, TDPSI drugs may be used at very low and safe doses with the primary goal of turning off those oncoproteins involved in chemoresistance; the cancer cells will then become highly sensitive to existing chemotherapeutic agents. In the latter scenario, TDPSI will be used as a priming agent, or adjuvant therapy, to overcome or avoid resistance to chemotherapy.

### F. Pharmaceutical Compositions

Certain embodiments of the present invention are directed to TDIs and pharmaceutical compositions comprising them as described herein for delivery of iron to tumors specifically, either intracellularly or extracellularly to improve the efficacy of radiation and chemotherapy, to enhance the detection of cancer, and diagnose cancer more easily. Other embodiments are directed to TDPSIs and pharmaceutical compositions and kits comprising them for delivery of potent and specific PSIs to cancer cells and ultimately treatment of cancer.

The therapeutic agents are generally administered in an amount sufficient to treat or prevent an enumerated disease (e.g., CD-22, CD-30, CD-33, CD-79 or HER-2 expressing cancers). The pharmaceutical compositions of the invention provide a therapeutic amount of the active agents effective to treat or prevent an enumerated disease or disorder.

Active agents of the invention may be chemically modified to facilitate uptake using methods known in the art.

The term “administer” is used in its broadest sense and includes any method of introducing the compositions of the present invention into a subject. Administration of an agent “in combination with” includes parallel administration of two agents to the patient over a period of time, co-administration (in which the agents are administered at approximately the same time, e.g., within about a few minutes to a few hours of one another), and co-formulation (in which the agents are combined or compounded into a single dosage form suitable for oral, subcutaneous or parenteral administration).

The pharmaceutical compositions of the present invention may be administered in a number of ways depending upon whether local (to the tumors) or systemic treatment if desired alone or in combination with an anti-cancer agent. Anti-cancer agents known in the art are not limited to the following in Table 4.

TABLE 4
Anti-Cancer Agents

Abiraterone Acetate
Abitrexate (Methotrexate)
Abraxane (Paclitaxel Albumin-stabilized Nanoparticle Formulation)
ABVD
ABVE
ABVE-PC
AC
AC-T
Adcetris (Brentuximab Vedotin)
ADE
Ado-Trastuzumab Emtansine
Adriamycin (Doxorubicin Hydrochloride)
Adrucil (Fluorouracil)
Afatinib Dimaleate
Afinitor (Everolimus)
Aldara (Imiquimod)
Aldesleukin
Alemtuzumab
Alimta (Pemetrexed Disodium)
Aloxi (Palonosetron Hydrochloride)
Ambochlorin (Chlorambucil)
Amboclorin (Chlorambucil)
Aminolevulinic Acid
Anastrozole
Aprepitant
Aredia (Pamidronate Disodium)
Arimidex (Anastrozole)
Aromasin (Exemestane)
Arranon (Nelarabine)
Arsenic Trioxide
Arzerra (Ofatumumab)
Asparaginase Erwinia chrysanthemi
Avastin (Bevacizumab)
Axitinib
Azacitidine
BEACOPP
Becenum (Carmustine)
Beleodaq (Belinostat)
Belinostat
Bendamustine Hydrochloride
BEP
Bevacizumab
Bexarotene
Bexxar (Tositumomab and I 131 Iodine Tositumomab)
Bicalutamide
BiCNU (Carmustine)
Bleomycin
Blinatumomab
Blincyto (Blinatumomab)
Bortezomib
Bosulif (Bosutinib)
Bosutinib
Brentuximab Vedotin
Busulfan
Busulfex (Busulfan)
Cabazitaxel
Cabozantinib-S-Malate
CAF
Campath (Alemtuzumab)
Camptosar (Irinotecan Hydrochloride)
Capecitabine
CAPOX
Carboplatin
CARBOPLATIN-TAXOL
Carfilzomib
Carmubris (Carmustine)
Carmustine
Carmustine Implant
Casodex (Bicalutamide)
CeeNU (Lomustine)
Ceritinib
Cerubidine (Daunorubicin Hydrochloride)
Cervarix (Recombinant HPV Bivalent Vaccine)
Cetuximab
Chlorambucil
CHLORAMBUCIL-PREDNISONE
CHOP
Cisplatin
Clafen (Cyclophosphamide)
Clofarabine
Clofarex (Clofarabine)
Clolar (Clofarabine)
CMF
Cometriq (Cabozantinib-S-Malate)
COPP
COPP-ABV
Cosmegen (Dactinomycin)
Crizotinib
CVP
Cyclophosphamide
Cyfos (Ifosfamide)
Cyramza (Ramucirumab)
Cytarabine
Cytarabine, Liposomal
Cytosar-U (Cytarabine)
Cytoxan (Cyclophosphamide)
Dabrafenib
Dacarbazine
Dacogen (Decitabine)
Dactinomycin
Dasatinib
Daunorubicin Hydrochloride
Decitabine
Degarelix
Denileukin Diftitox
Denosumab
Dinutuximab
DepoCyt (Liposomal Cytarabine)
DepoFoam (Liposomal Cytarabine)
Dexrazoxane Hydrochloride
Docetaxel
Doxil (Doxorubicin Hydrochloride Liposome)
Doxorubicin Hydrochloride
Doxorubicin Hydrochloride Liposome
Dox-SL (Doxorubicin Hydrochloride Liposome)
DTIC-Dome (Dacarbazine)
Efudex (Fluorouracil)
Elitek (Rasburicase)
Ellence (Epirubicin Hydrochloride)
Eloxatin (Oxaliplatin)
Eltrombopag Olamine
Emend (Aprepitant)
Enzalutamide
Epirubicin Hydrochloride
EPOCH
Erbitux (Cetuximab)
Eribulin Mesylate
Erivedge (Vismodegib)
Erlotinib Hydrochloride
Erwinaze (Asparaginase Erwinia chrysanthemi)
Etopophos (Etoposide Phosphate)
Etoposide
Etoposide Phosphate
Evacet (Doxorubicin Hydrochloride Liposome)
Everolimus
Evista (Raloxifene Hydrochloride)
Exemestane
Fareston (Toremifene)
Farydak (Panobinostat)
Faslodex (Fulvestrant)
FEC
Femara (Letrozole)
Filgrastim
Fludara (Fludarabine Phosphate)
Fludarabine Phosphate
Fluoroplex (Fluorouracil)
Fluorouracil
Folex (Methotrexate)
Folex PFS (Methotrexate)
FOLFIRI
FOLFIRI-BEVACIZUMAB
FOLFIRI-CETUXIMAB
FOLFIRINOX
FOLFOX
Folotyn (Pralatrexate)
FU-LV
Fulvestrant
Gardasil (Recombinant HPV Quadrivalent Vaccine)
Gardasil 9 (Recombinant HPV Nonavalent Vaccine)
Gazyva (Obinutuzumab)
Gefitinib
Gemcitabine Hydrochloride
GEMCITABINE-CISPLATIN
GEMCITABINE-OXALIPLATIN
Gemtuzumab Ozogamicin
Gemzar (Gemcitabine Hydrochloride)
Gilotrif (Afatinib Dimaleate)
Gleevec (Imatinib Mesylate)
Gliadel (Carmustine Implant)
Gliadel wafer (Carmustine Implant)
Glucarpidase
Goserelin Acetate
Halaven (Eribulin Mesylate)
Herceptin (Trastuzumab)
HPV Bivalent Vaccine, Recombinant
HPV Nonavalent Vaccine, Recombinant
HPV Quadrivalent Vaccine, Recombinant
Hycamtin (Topotecan Hydrochloride)
Hyper-CVAD
Ibrance (Palbociclib)
Ibritumomab Tiuxetan
Ibrutinib
ICE
Iclusig (Ponatinib Hydrochloride)
Idamycin (Idarubicin Hydrochloride)
Idarubicin Hydrochloride
Idelalisib
Ifex (Ifosfamide)
Ifosfamide
Ifosfamidum (Ifosfamide)
Imatinib Mesylate
Imbruvica (Ibrutinib)
Imiquimod
Inlyta (Axitinib)
Intron A (Recombinant Interferon Alfa-2b)
Iodine 131 Tositumomab and Tositumomab
Ipilimumab
Iressa (Gefitinib)
Irinotecan Hydrochloride
Istodax (Romidepsin)
Ixabepilone
Ixempra (Ixabepilone)
Jakafi (Ruxolitinib Phosphate)
Jevtana (Cabazitaxel)
Kadcyla (Ado-Trastuzumab Emtansine)
Keoxifene (Raloxifene Hydrochloride)
Kepivance (Palifermin)
Keytruda (Pembrolizumab)
Kyprolis (Carfilzomib)
Lanreotide Acetate
Lapatinib Ditosylate
Lenalidomide
Lenvatinib Mesylate
Lenvima (Lenvatinib Mesylate)
Letrozole
Leucovorin Calcium
Leukeran (Chlorambucil)
Leuprolide Acetate
Levulan (Aminolevulinic Acid)
Linfolizin (Chlorambucil)
LipoDox (Doxorubicin Hydrochloride Liposome)
Liposomal Cytarabine
Lomustine
Lupron (Leuprolide Acetate)
Lupron Depot (Leuprolide Acetate)
Lupron Depot-Ped (Leuprolide Acetate)
Lupron Depot-3 Month (Leuprolide Acetate)
Lupron Depot-4 Month (Leuprolide Acetate)
Lynparza (Olaparib)
Margibo (Vincristine Sulfate Liposome)
Matulane (Procarbazine Hydrochloride)
Mechlorethamine Hydrochloride
Megace (Megestrol Acetate)
Megestrol Acetate
Mekinist (Trametinib)
Mercaptopurine
Mesna
Mesnex (Mesna)
Methazolastone (Temozolomide)
Methotrexate
Methotrexate LPF (Methotrexate)
Mexate (Methotrexate)
Mexate-AQ (Methotrexate)
Mitomycin C
Mitoxantrone Hydrochloride
Mitozytrex (Mitomycin C)
MOPP
Mozobil (Plerixafor)
Mustargen (Mechlorethamine Hydrochloride)
Mutamycin (Mitomycin C)
Myleran (Busulfan)
Mylosar (Azacitidine)
Mylotarg (Gemtuzumab Ozogamicin)
Nanoparticle Paclitaxel (Paclitaxel Albumin-stabilized Nanoparticle
Formulation)
Navelbine (Vinorelbine Tartrate)
Nelarabine
Neosar (Cyclophosphamide)
Neupogen (Filgrastim)
Nexavar (Sorafenib Tosylate)
Nilotinib
Nivolumab
Nolvadex (Tamoxifen Citrate)
Nplate (Romiplostim)
Obinutuzumab
OEPA
Ofatumumab
OFF
Olaparib
Omacetaxine Mepesuccinate
Oncaspar (Pegaspargase)
Ontak (Denileukin Diftitox)
Opdivo (Nivolumab)
OPPA
Oxaliplatin
Paclitaxel
Paclitaxel Albumin-stabilized Nanoparticle Formulation
PAD
Palbociclib
Palifermin
Palonosetron Hydrochloride
Pamidronate Disodium
Panitumumab
Panobinostat
Paraplat (Carboplatin)
Paraplatin (Carboplatin)
Pazopanib Hydrochloride
Pegaspargase
Peginterferon Alfa-2b
PEG-Intron (Peginterferon Alfa-2b)
Pembrolizumab
Pemetrexed Disodium
Perjeta (Pertuzumab)
Pertuzumab
Platinol (Cisplatin)
Platinol-AQ (Cisplatin)
Plerixafor
Pomalidomide
Pomalyst (Pomalidomide)
Ponatinib Hydrochloride
Pralatrexate
Prednisone
Procarbazine Hydrochloride
Proleukin (Aldesleukin)
Prolia (Denosumab)
Promacta (Eltrombopag Olamine)
Provenge (Sipuleucel-T)
Purinethol (Mercaptopurine)
Purixan (Mercaptopurine)
Radium 223 Dichloride
Raloxifene Hydrochloride
Ramucirumab
Rasburicase
R-CHOP
R-CVP
Recombinant Human Papillomavirus (HPV) Bivalent Vaccine
Recombinant Human Papillomavirus (HPV) Nonavalent Vaccine
Recombinant Human Papillomavirus (HPV) Quadrivalent Vaccine
Recombinant Interferon Alfa-2b
Regorafenib
R-EPOCH
Revlimid (Lenalidomide)
Rheumatrex (Methotrexate)
Rituxan (Rituximab)
Rituximab
Romidepsin
Romiplostim
Rubidomycin (Daunorubicin Hydrochloride)
Ruxolitinib Phosphate
Sclerosol Intrapleural Aerosol (Talc)
Siltuximab
Sipuleucel-T
Somatuline Depot (Lanreotide Acetate)
Sorafenib Tosylate
Sprycel (Dasatinib)
STANFORD V
Sterile Talc Powder (Talc)
Steritalc (Talc)
Stivarga (Regorafenib)
Sunitinib Malate
Sutent (Sunitinib Malate)
Sylatron (Peginterferon Alfa-2b)
Sylvant (Siltuximab)
Synovir (Thalidomide)
TAC
Tafinlar (Dabrafenib)
Talc
Tamoxifen Citrate
Tarabine PFS (Cytarabine)
Tarceva (Erlotinib Hydrochloride)
Targretin (Bexarotene)
Tasigna (Nilotinib)
Taxol (Paclitaxel)
Taxotere (Docetaxel)
Temodar (Temozolomide)
Temozolomide
Temsirolimus
Thalidomide
Thalomid (Thalidomide)
Thiotepa
Toposar (Etoposide)
Topotecan Hydrochloride
Toremifene
Torisel (Temsirolimus)
Tositumomab and I 131 Iodine Tositumomab
Totect (Dexrazoxane Hydrochloride)
TPF
Trametinib
Trastuzumab
Treanda (Bendamustine Hydrochloride)
Trisenox (Arsenic Trioxide)
Tykerb (Lapatinib Ditosylate)
Unituxin (Dinutuximab)
Vandetanib
VAMP
Vectibix (Panitumumab)
VeIP
Velban (Vinblastine Sulfate)
Velcade (Bortezomib)
Velsar (Vinblastine Sulfate)
Vemurafenib
VePesid (Etoposide)
Viadur (Leuprolide Acetate)
Vidaza (Azacitidine)
Vinblastine Sulfate
Vincasar PFS (Vincristine Sulfate)
Vincristine Sulfate
Vincristine Sulfate Liposome
Vinorelbine Tartrate
VIP
Vismodegib
Voraxaze (Glucarpidase)
Vorinostat
Votrient (Pazopanib Hydrochloride)
Wellcovorin (Leucovorin Calcium)
Xalkori (Crizotinib)
Xeloda (Capecitabine)
XELIRI
XELOX
Xgeva (Denosumab)
Xofigo (Radium 223 Dichloride)
Xtandi (Enzalutamide)
Yervoy (Ipilimumab)
Zaltrap (Ziv-Aflibercept)
Zelboraf (Vemurafenib)
Zevalin (Ibritumomab Tiuxetan)
Zinecard (Dexrazoxane Hydrochloride)
Ziv-Aflibercept
Zoladex (Goserelin Acetate)
Zoledronic Acid
Zolinza (Vorinostat)
Zometa (Zoledronic Acid)
Zydelig (Idelalisib)
Zykadia (Ceritinib)
Zytiga (Abiraterone Acetate)

Administration can be oral, intravenous, parenteral, intra-arterial, subcutaneous, intraperitoneal or intramuscular injection or infusion; or intracranial, e.g., intrathecal or intraventricular, administration. In recent years there has been a tendency towards the development of controlled release dosage forms that will provide therapy over an extended period of time. Normally this would be once a day and it is believed that such a change in dosage regimen will reduce adverse reactions and side effects and also improve patient compliance. The use of synthetic polymers that may have muco- or bio-adhesive properties has been investigated and is disclosed in WO 85/02092.

In some embodiments, a slow release preparation comprising the active agents is formulated. It is desirable to prolong delivery with these slow release preparations so that the drug may be released at a desired rate over this prolonged period. By extending the period, the drug can if required be released more slowly, which may lead to less severe adverse reactions and side effects. The preparation of sustained, controlled, delayed or anyhow modified release form can be carried out according to different known techniques: 1. The use of inert matrices, in which the main component of the matrix structure opposes some resistance to the penetration of the solvent due to the poor affinity towards aqueous fluids; such property being known as lipophilia; 2. The use of hydrophilic matrices, in which the main component of the matrix structure opposes high resistance to the progress of the solvent, in that the presence of strongly hydrophilic groups in its chain, mainly branched, remarkably increases viscosity inside the hydrated layer; and 3. The use of bioerodible matrices, which are capable of being degraded by the enzymes of some biological compartment. See. U.S. Pat. No. 7,431,943.

The term “slow release” refers to the release of a drug from a polymeric drug delivery system over a period of time that is more than one day wherein the active agent is formulated in a polymeric drug delivery system that releases effective concentrations of the drug. Drug delivery systems may include a plurality of polymer particles containing active drug material, each of the particles preferably having a size of 20 microns or less, and incorporating on the outer surface of at least some of the particles a bioadhesive material derived from a bacterium. Such drug delivery systems have been described in U.S. Pat. No. 6,355,276. The use of these microorganisms in the design allow for a controlled release dosage form with extended gastrointestinal residence.

In certain embodiments, dosage forms of the compositions of the present invention include, but are not limited to, implantable depot systems. In one embodiment, the depot system includes a three-dimensional matrix.

Self emulsifying microemulsion drug delivery systems (SMEDDS) are known in the art as effective delivery systems into the G.I. tract. See U.S. Patent Application 2001/00273803. The term SMEDDS is defined as isotropic mixtures of oil, surfactant, cosurfactant and drug that rapidly form oil in water microemulsion when exposed to aqueous media or gastrointestinal fluid under conditions of gentle agitation or digestive motility that would be encountered in the G.I. tract.

Thermostable nanoparticles may be contained in a drug delivery system targeted for the G.I. tract. See U.S. Patent Application 2000/60193787. These drug delivery systems may include at least one type of biodegradable and/or bioresorbable nanoparticle and at least one drug that possesses at least one of the following properties: emulsifier or mucoadhesion. The drug may substantially cover the surface of the nanoparticle.

In certain embodiments, the pharmaceutical compositions of the present invention comprise about 0.1 mg to 5 g, about 0.5 mg to about 1 g, about 1 mg to about 750 mg, about 5 mg to about 500 mg, or about 10 mg to about 100 mg of therapeutic agent.

Active agents can be administered as a single treatment or, preferably, can include a series of treatments that continue at a frequency and for duration of time that causes one or more symptoms of the enumerated disease to be reduced or ameliorated, or that achieves the desired effect including reducing tumor burden or metastasis.

It is understood that the appropriate dose of an active agent depends upon a number of factors within the ken of the ordinarily skilled physician, veterinarian, or researcher. The dose(s) vary, for example, depending upon the identity, size, and condition of the subject or sample being treated, further depending upon the route by which the composition is to be administered, and the effect which the practitioner desires the an active agent to have. It is furthermore understood that appropriate doses of an active agent depend upon the potency with respect to the expression or activity to be modulated. Such appropriate doses may be determined using the assays described herein. When one or more of these active agents are to be administered to an animal (e.g., a human), a relatively low dose may be prescribed at first, with the dose subsequently increased until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The therapeutic agent can be formulated with an acceptable carrier using methods well known in the art. The actual amount of therapeutic agent will necessarily vary according to the particular formulation, route of administration, and dosage of the pharmaceutical composition, the specific nature of the condition to be treated, and possibly the individual subject. The dosage for the pharmaceutical compositions of the present invention can range broadly depending upon the desired effects, the therapeutic indication, and the route of administration, regime, and purity and activity of the composition.

A suitable subject, preferably a human, can be an individual or animal that is suspected of having, has been diagnosed as having, or is at risk of developing an enumerated disease, and like conditions as can be determined by one knowledgeable in the art.

Techniques for formulation and administration can be found in “Remington: The Science and Practice of Pharmacy” (20th edition, Gennaro (ed.) and Gennaro, Lippincott, Williams & Wilkins, 2000), incorporated herein by reference.

Active agents may be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylene diamine tetra acetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where the therapeutic agents are water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It should be stable under the conditions of manufacture and storage and should be preserved against the contaminating action of microorganisms such as bacteria and fungi.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active agent in the required amount in an appropriate solvent with one or a combination of the ingredients enumerated above, as required, followed by filter sterilization. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. Depending on the specific conditions being treated, pharmaceutical compositions of the present invention for treatment of atherosclerosis or the other elements of metabolic syndrome can be formulated and administered systemically or locally. Techniques for formulation and administration can be found in “Remington: The Science and Practice of Pharmacy” (20th edition, Gennaro (ed.) and Gennaro, Lippincott, Williams & Wilkins, 2000). For oral administration, the agent can be contained in enteric forms to survive the stomach or further coated or mixed to be released in a particular region of the GI tract by known methods. For the purpose of oral therapeutic administration, the active agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL® or corn starch; a lubricant such as magnesium stearate or STEROTES® a glidant such as colloidal silicon dioxide; a sweetening agent such as sucrose or saccharin; or a flavoring agent such as peppermint, methyl salicylate, or orange flavoring.

Systemic administration can also be by transmucosal means to the intestinal or colon. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams as generally known in the art.

In one embodiment, the active agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells with, e.g., monoclonal antibodies) can also be used as pharmaceutically acceptable carriers. These can be prepared according to methods known to those skilled in the art, for example, as described in U.S. Pat. No. 4,522,811.

### F. Kits

The present invention provides a kit of manufacture, which may be used to contain a pharmaceutical composition. In one embodiment, an article of manufacture (i.e., kit) according to the present invention includes a TDI drug or TDPSI drug and one or more reagent. In another embodiment, the present kit contains a TDI drug or TDPSI drug and an anti-cancer agent and one or more reagent.

Kits provided herein may also include instructions, such as a package insert having instructions.

In another embodiment, the kit further comprises reagents used in the preparation of the sample to be tested (e.g. lysis buffer).

TABLE 5
Examples of Linkers
Linker

| Type | Sequence^{a} |
|---|---|
| flexible | (GGGGS)₃ |
| flexible | (GGGGS)₃ |
| flexible | (GGGGS)₃ |
| flexible | (Gly)₈ |
| flexible | (Gly)₆ |
| rigid | (EAAAK)₃ |
| rigid | (EAAAK)ₙ (n = 1-3) |
| rigid | A(EAAAK)₄ALEA(EAAAK)₄A |
| rigid | A(EAAAK)₄ALEA(EAAAK)₄A |
| flexible | (GGGGS)₃ |
| rigid | A(EAAAK)₄ALEA(EAAAK)₄A |
| rigid | A(EAAAK)₄ALEA(EAAAK)₄A |
| flexible | GGGGS |
| rigid | PAPAP |
| rigid | AEAAAKEAAAKA |
| flexible | (GGGGS)ₙ (n = 1, 2, 4) |
| rigid | (Ala-Pro)ₙ (10-34 aa) |
| cleavable | disulfide |
| cleavable | disulfide |
| cleavable | VSQTSKLTR↓AETVFPDV^{b} |
| cleavable | PLG ↓ LWA^{c} |
| cleavable | RVL↓AEA; EDVVCC↓SMSY; |
| | GGIEGR↓GS^{c} |
| cleavable | TRHRQPR↓GWE; |
| | AGNRVRR↓SVG; |
| | RRRRRRR↓R↓R^{d} |
| cleavable | GFLG↓^{e} |
| dipeptide | LE |
| rigid | A(EAAAK)₄ALEA(EAAAK)₄A |
| cleavable | Disulfide |

^{a}Protease sensitive cleavage sites are indicated with “↓”
^{b}Factor XIa/FVIIa sensitive cleavage
^{c}Matrix metalloprotease-1 sensitive cleavage sequences, one example provided here
^{d}HIV PR (HIV-1 protease); NS3 protease (HCV protease); Factor Xa sensitive cleavage, respectively
^{e}Furin sensitive cleavage
^{f}Cathepsin B sensitive cleavage

1-29. (canceled)

30. A drug for delivery to a tumor, comprising a heme fused to an antibody, aptamer or fragment thereof that contains an antigenic binding site specific for a cancer cell surface antigen.

31. The drug of claim 30 wherein the cancer cell surface antigen is selected from the group consisting of CD19, CD20, CD22, CD30, CD33, CD37, CD38, CD39, CD52, CD79, EGFR, and HER2.

32. The drug of claim 31 wherein the cancer cell surface antigen is selected from the group consisting of CD22, CD30, CD33, and CD79.

33. The drug of claim 30 wherein the antibody is a monoclonal antibody.

34. The drug of claim 30 which comprises two antibodies, aptamers or fragments thereof that contain an antigenic binding site, wherein each antibody is specific for a different cancer cell surface antigen.

35. The drug of claim 30 which further comprises a peptide linker that optionally is cleavable inside a cancer cell.

36. A pharmaceutical composition comprising a pharmaceutical carrier and a therapeutically effective amount of the drug of claim 30.

37. A method of increasing iron content in tumor tissue to enhance detection of cancer in a subject in need thereof, comprising administering the pharmaceutical composition of claim 36 to the subject.

38. The method of claim 37 wherein the tumor is selected from a lymphoma and a breast cancer.

39. The method of claim 37 wherein the tumor expresses CD22, CD30, CD79 or HER2.

40. The method of claim 37 which improves the efficacy of radiation, radio-diagnosis, chemotherapy, or a combination thereof.

41. A drug for delivery to a tumor, comprising a protein synthesis inhibitor drug fused to an antibody, aptamer or fragment thereof that contains an antigenic binding site specific for a cancer cell surface antigen.

42. The drug of claim 41 wherein the cancer cell surface antigen is selected from CD30 and HER2.

43. The drug of claim 41 wherein the antibody is a monoclonal antibody.

44. The drug of claim 41 which comprises two antibodies, aptamers or fragments thereof that contain an antigenic binding site, wherein each antibody is specific for a different cancer cell surface antigen.

45. The drug of claim 41 which further comprises a peptide linker that optionally is cleavable inside a cancer cell.

46. A pharmaceutical composition comprising a pharmaceutical carrier and a therapeutically effective amount of the drug of claim 41.

47. A method of treating cancer in a subject in need thereof, comprising administering the pharmaceutical composition of claim 46 to the subject.

48. The method of claim 47 wherein the cancer expresses CD30 or HER2.

49. A method comprising:
(a) administering to a subject suspected of having cancer the drug of claim 30 to enhance detection of cancer;
(b) performing an MRI, CT, or PET scan on the subject; and
(c) diagnosing the subject as having cancer.
